(19) United States
(12) Patent Application Publication
(10) Pub. No.: US 2008/0140155 A1
(43) Pub. Date: Jun. 12, 2008

(54) Excessive fibrous capsule formation and capsular contracture apparatus and method for using same

(76) Inventors: Arthur A. Pilla, Oakland, NJ (US); Andre DiMino, Woodcliff Lake, NJ (US)

Correspondence Address: LEN TAYLOR, PATENT ATTORNEY, 261 DAVENPORT STREET, SOMERVILLE, NJ, 08876, US

(21) Appl. No.: 11/977,043

(22) Filed: Oct. 22, 2007

Related U.S. Application Data
(60) Provisional application No. 60/852,927, filed on Oct. 20, 2006.

(51) Int. Cl. A61N 1/04

(52) U.S. Cl. 607/50

(57) ABSTRACT

An apparatus comprising an electromagnetic signal generating means for emitting signals comprising bursts of at least one of sinusoidal, rectangular, chaotic, and random waveforms, having a frequency content in a range of about 0.01 Hz to about 100 MHz at about 1 to about 100,000 waveforms per second, having a burst duration of 1 usec to 100 msec, and having a burst repetition rate from about 0.01 to about 1000 bursts/second, wherein the waveforms are adapted to a frequency response of a fibrous capsule formation and capsular contracture target pathway structure and to have sufficient signal to noise ratio of at least about 0.2 in respect of a given fibrous capsule formation and capsular contracture target pathway structure to modulate at least one of ion and ligand interactions in that fibrous capsule formation and capsular target pathway structure wherein the signal to noise ratio is evaluated by calculating a frequency response of the impedance of the target path structure divided by a calculated frequency response of baseline thermal fluctuations in voltage across the target path structure, an electromagnetic signal coupling means wherein the coupling means comprises at least one of an inductive coupling means and a capacitive coupling means, connected to the electromagnetic signal generating means for delivering the electromagnetic signal to the fibrous capsule formation and capsular contracture target pathway structure, and a garment wherein the electromagnetic signal generating means and electromagnetic signal coupling means are incorporated into the garment.

This application claims the benefit of U.S. Provisional Application 60/852,927 filed Oct. 20, 2006, herein incorporated by reference in its entirety.

### BACKGROUND OF THE INVENTION

### 1. Field of the Invention

This invention pertains generally to an electromagnetic coil apparatus and a method that configures and delivers electromagnetic signals to promote cell and tissue growth, repair, and maintenance. The electromagnetic environment of living tissues, cells, and molecules is altered by the electromagnetic signals generated by an embodiment of the present invention to achieve a therapeutic or wellness effect. Alteration of the electromagnetic environment can be particularly effective for alleviating pain and discomfort in individuals having capsular contracture or excessive fibrous capsule formation associated with any surgically implanted device. The invention also relates to a method of modification of cellular and tissue growth, repair, maintenance and general behavior by the application of encoded electromagnetic information. More particularly, this invention provides for an application of highly specific electromagnetic frequency (“EMF”) signal patterns to excessive fibrous capsule tissue by non-invasive reactive coupling of encoded electromagnetic information. Such application of electromagnetic waveforms to human and animal fibrous capsule formation and capsular contracture target pathway structures such as cells, organs, tissues and molecules, can reduce the pain and edema associated with capsular contracture, can increase blood flow, neovascularization, vascularogenesis, and angiogenesis and can augment the release of growth factors and cytokines related to the prophylactic and a posteriori treatment of excessive fibrous capsule formation.

The present invention further relates to altering the cellular and molecular mechanisms of excessive fibrous capsule formation and to control capsular contracture generally associated with post surgical complications of implants such as breast augmentation.

Capsular contracture is a painful inflammatory condition which can occur at any time post surgically but usually occurs within the first several months after surgery. Capsular contracture is the most common complication of breast augmentation surgery but also can occur with other surgically implanted devices. At the time of initial breast augmentation surgery, a pocket is made for a breast implant in tissue covering the chest. During the healing process a capsule that is comprised of fibrous tissue forms. The body is genetically programmed to counteract that formation by attempting to shrink the scar tissue to a certain degree. Under normal circumstances, the pocket remains open thus allowing the implant to look and feel natural. However in a certain number of cases, the capsule will tighten thereby causing pressure by restricting the space for the implant. Furthermore this causes the implant to feel hard and rigid with concomitant distortion of the appearance of the breast. In later stages the implant feels extremely firm and may take on an unnatural “ball like” appearance. The present invention produces a physiological effect in the tissue of a capsular contracture. The physiological effect causes revascularization and inter-cellular modification tissue, to reduce in hardness and prevalence thereby reducing pain and discomfort for a patient. Waveforms produced by the within invention accelerate or modify a number of physiological cascades that either alleviate the propensity of the capsule to compress or harden, or produce a reduction in the existing capsule involvement with the physical area at which the waveforms have been applied to. In particular a pulsing electromagnetic field (“PEMF”) signal can enhance production of nitric oxide (“NO”) via modulation of Calcium (“Ca²⁺”) binding to calmodulin (“CaM”). This in turn can inhibit inflammatory leukotrienes that reduce the inflammatory process leading to excessive fibrous capsule formation. At present, pharmacologic agents targeted to inhibit leukotrienes are employed for treating capsular contracture with limited success. Prophylactic use of the within invention prior to device implant in individuals that are deemed susceptible to capsular contracture formation may prevent or reduce the formation of excessive fibrous tissue.

An advantageous result of the within invention is that by applying a high spectral density voltage envelope as the modulating or pulse-burst defining parameter, the power requirement for such increased duration pulse bursts can be significantly lower than that of shorter pulse bursts containing pulses within the same frequency range. This is due to more efficient matching of the frequency components to relevant cellular and molecular processes. Accordingly the dual advantages of enhanced transmitted dosimetry to the relevant dielectric pathways and of decreased power requirements are achieved. This allows for the implementation of the within invention in an easily transportable unit for ease of application on capsular contracture patients.

Therefore, a need exists for an apparatus and a method that effectively accelerates or modifies a number of physiological cascades that alleviate the propensity of the capsule to compress or harden, that reduce excessive fibrous capsule formation, and that produce a reduction in the existing capsule involvement within the physical area to which the waveforms have been applied.

### SUMMARY OF THE INVENTION

The apparatus and method according to present invention, comprise delivering electromagnetic signals to fibrous capsule formation and capsular contracture target pathway structures, such as capsular molecules, capsular cells, capsular tissues, and capsular organs for alleviation of the propensity of a capsule to compress or harden, for reduction of excessive fibrous capsule formation, and for reduction in existing capsule involvement with a physical area of a body. An embodiment according to the present invention utilizes SNR and Power SNR approaches to configure bioeffective waveforms and incorporates miniaturized circuitry and lightweight flexible coils. This advantageously allows a device that utilizes the SNR and Power SNR approaches, miniaturized circuitry, and lightweight flexible coils to be completely portable and if desired to be constructed as disposable.

An apparatus comprising an electromagnetic signal generating means for emitting signals comprising bursts of at least one of sinusoidal, rectangular, chaotic, and random waveforms, having a frequency content in a range of about 0.01 Hz to about 100 MHz at about 1 to about 100,000 waveforms per second, having a burst duration from about 1 usec to about 100 msec, and having a burst repetition rate from about 0.01 to about 1000 bursts/second, wherein the waveforms are adapted to have sufficient signal to noise ratio of at least about 0.2 in respect of a given fibrous capsule formation and capsular contracture target pathway structure to modulate at least one of ion and ligand interactions in that fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure, an electromagnetic signal coupling means wherein the coupling means comprises at least one of an inductive coupling means and a capacitive coupling means, connected to the electromagnetic signal generating means for delivering the electromagnetic signal to the fibrous capsule formation and capsular contracture target pathway structure, and a garment wherein the electromagnetic signal generating means and electromagnetic signal coupling means are incorporated into the garment.

An apparatus comprising a waveform configuration means for configuring at least one waveform to have sufficient signal to noise ratio or power signal to noise ratio of at least about 0.2, to modulate at least one of ion and ligand interactions whereby the at least one of ion and ligand interactions are detectable in a fibrous capsule formation and capsular contracture target pathway structure above baseline thermal fluctuations in voltage and electrical impedance at the fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure, a coupling device connected to the waveform configuration means by at least one connecting means for generating an electromagnetic signal from the configured at least one waveform and for coupling the electromagnetic signal to the fibrous capsule formation and capsular contracture target pathway structure whereby the at least one of ion and ligand interactions are modulated, and a garment incorporating the waveform configuration means, the at least one connecting means, and the coupling device.

A method comprising establishing baseline thermal fluctuations in voltage and electrical impedance at a fibrous capsule formation and capsular contracture target pathway structure depending on a state of the fibrous capsule tissue, evaluating a signal to noise ratio by calculating a frequency response of the impedance of the target pathway structure divided by a calculated frequency response of baseline thermal fluctuations in voltage across the target pathway structure, configuring at least one waveform to have sufficient signal to noise ratio of at least about 0.2 to modulate at least one of ion and ligand interactions whereby the at least one of ion and ligand interactions are detectable in the fibrous capsule formation and capsular contracture target pathway structure above the evaluated baseline thermal fluctuations in voltage, generating an electromagnetic signal from the configured at least one waveform; and coupling the electromagnetic signal to the fibrous capsule formation and capsular contracture target pathway structure using a coupling device.

“About” for purposes of the invention means a variation of plus or minus 50%.

The above and yet other aspects and advantages of the present invention will become apparent from the hereinafter set forth Brief Description of the Drawings and Detailed Description of the Invention.

### BRIEF DESCRIPTION OF THE DRAWINGS

Methods and apparatus that are particular embodiments of the invention will now be described, by way of example, with reference to the accompanying diagrammatic drawings:

FIG. 1 is a flow diagram of a method for altering fibrous capsule formation and capsular contracture according to an embodiment of the present invention;

FIG. 2 is a view of an apparatus for application of electromagnetic signals according to an embodiment of the present invention;

FIG. 3 is a block diagram of miniaturized circuitry according to an embodiment of the present invention;

FIG. 4 depicts a waveform delivered to a capsule formation and capsule contracture target pathway structure according to an embodiment of the present invention;

FIG. 5 is a view of inductors placed in a vest according to an embodiment of the present invention;

FIG. 6 is a bar graph illustrating myosin phosphorylation for a PMF signal configured according to an embodiment of the present invention; and

FIG. 7 is a bar graph illustrating SNR signal effectiveness in a cell model of inflammation.

### DETAILED DESCRIPTION OF THE INVENTION

Induced time-varying currents from PEMF or PRF devices flow in a fibrous capsule formation and capsular contracture target pathway structure such as a molecule, cell, tissue, and organ, and it is these currents that are a stimulus to which cells and tissues can react in a physiologically meaningful manner. The electrical properties of a fibrous capsule formation and capsular contracture target pathway structure affect levels and distributions of induced current. Molecules, cells, tissue, and organs are all in an induced current pathway such as cells in a gap junction contact. Ion or ligand interactions at binding sites on macromolecules that may reside on a membrane surface are voltage dependent chemical processes, that is electrochemical, that can respond to an induced electromagnetic field (“E”). Induced current arrives at these sites via a surrounding ionic medium. The presence of cells in a current pathway causes an induced current (“J”) to decay more rapidly with time (“J(t)”). This is due to an added electrical impedance of cells from membrane capacitance and ion binding time constants of binding and other voltage sensitive membrane processes such as membrane transport. Knowledge of ion binding time constants allows SNR to be evaluated for any EMF signal configuration. Preferably ion binding time constants in the range of about 1 to about 100 msec are used.

Equivalent electrical circuit models representing various membrane and charged interface configurations have been derived. For example, in Calcium (“Ca²⁺”) binding, the change in concentration of bound Ca²⁺ at a binding site due to induced E may be described in a frequency domain by an impedance expression such as:

$${Z}_{b} ( ω ) = {R}_{ion} + \frac{1}{i ω {C}_{ion}}$$

which has the form of a series resistance-capacitance electrical equivalent circuit. Where ω is angular frequency defined as 2nf, where f is frequency, i=−1^{1/2}, Zb(ω) is the binding impedance, and Rᵢₒₙ and Cᵢₒₙ are equivalent binding resistance and capacitance of an ion binding pathway. The value of the equivalent binding time constant, τᵢₒₙ=RᵢₒₙCᵢₒₙ, is related to a ion binding rate constant, k_{b}, via τᵢₒₙ=RᵢₒₙCᵢₒₙ=1/k_{b}. Thus, the characteristic time constant of this pathway is determined by ion binding kinetics.

Induced E from a PEMF or PRF signal can cause current to flow into an ion binding pathway and affect the number of Ca²⁺ ions bound per unit time. An electrical equivalent of this is a change in voltage across the equivalent binding capacitance Cᵢₒₙ, which is a direct measure of the change in electrical charge stored by Cᵢₒₙ. Electrical charge is directly proportional to a surface concentration of Ca²⁺ ions in the binding site that is storage of charge is equivalent to storage of ions or other charged species on cell surfaces and junctions. Electrical impedance measurements, as well as direct kinetic analyses of binding rate constants, provide values for time constants necessary for configuration of a PMF waveform to match a bandpass of fibrous capsule formation and capsular contracture target pathway structures. This allows for a required range of frequencies for any given induced E waveform for optimal coupling to target impedance, such as bandpass.

Ion binding to regulatory molecules is a frequent EMF target, for example Ca²⁺ binding to calmodulin (“CaM”). Use of this pathway is based upon acceleration of tissue repair, for example bone repair, wound repair, hair repair, and repair of other molecules, cells, tissues, and organs that involves modulation of growth factors released in various stages of repair. Growth factors such as platelet derived growth factor (“PDGF”), fibroblast growth factor (“FGF”), and epidermal growth factor (“EGF”) are all involved at an appropriate stage of healing. Angiogenesis and neovascularization are also integral to tissue growth and repair and can be modulated by PMF. All of these factors are Ca/CaM-dependent.

Utilizing a Ca/CaM pathway a waveform can be configured for which induced power is sufficiently above background thermal noise power. Under correct physiological conditions, this waveform can have a physiologically significant bioeffect.

Application of a Power SNR model to Ca/CaM requires knowledge of electrical equivalents of Ca²⁺ binding kinetics at CaM. Within first order binding kinetics, changes in concentration of bound Ca²⁺ at CaM binding sites over time may be characterized in a frequency domain by an equivalent binding time constant, τᵢₒₙ=RᵢₒₙCᵢₒₙ, where R^{ion} and C^{ion} are equivalent binding resistance and capacitance of the ion binding pathway. τᵢₒₙ is related to a ion binding rate constant, k_{b}, via τᵢₒₙ=RᵢₒₙCᵢₒₙ=1/k_{b}. Published values for k_{b} can then be employed in a cell array model to evaluate SNR by comparing voltage induced by a PRF signal to thermal fluctuations in voltage at a CaM binding site. Employing numerical values for PMF response, such as Vmax=6.5×10−7 sec⁻¹, [Ca²⁺]=2.5 μM, KD=30 μM, [Ca²⁺CaM]=KD([Ca²⁺]+[CaM]), yields k_{b}=665 sec⁻¹ (τᵢₒₙ=1.5 msec). Such a value for τᵢₒₙ can be employed in an electrical equivalent circuit for ion binding while power SNR analysis can be performed for any waveform structure.

According to an embodiment of the present invention a mathematical model can be configured to assimilate that thermal noise which is present in all voltage dependent processes and represents a minimum threshold requirement to establish adequate SNR. Power spectral density, Sn(ω), of thermal noise can be expressed as:

Sₙ(ω)=4kT Re[Z_{M}(x,ω)]

where Z_{M}(x,ω) is electrical impedance of a fibrous capsule formation and capsular contracture target pathway structure, x is a dimension of a fibrous capsule formation and capsular contracture target pathway structure and Re denotes a real part of impedance of a fibrous capsule formation and capsular contracture target pathway structure. Z_{M}(x,ω) can be expressed as:

$${Z}_{M} ( x , ω ) = [ \frac{{R}_{e} + {R}_{i} + {R}_{g}}{γ} ] tan h ( γ x )$$

This equation clearly shows that electrical impedance of the fibrous capsule formation and capsular contracture target pathway structure, and contributions from extracellular fluid resistance (“Re”), intracellular fluid resistance (“Ri”) and intermembrane resistance (“Rg”) which are electrically connected to fibrous capsule formation and capsular contracture target pathway structures all contribute to noise filtering.

A typical approach to evaluation of SNR uses a single value of a root mean square (RMS) noise voltage. This is calculated by taking a square root of an integration of Sₙ(ω)=4kT Re[Z_{M}(x,ω)] over all frequencies relevant to either a complete membrane response, or to bandwidth of a fibrous capsule formation and capsular contracture target pathway structure. SNR can be expressed by a ratio:

$$SNR = \frac{\vert {V}_{M} ( ω ) \vert}{RMS}$$

where |V_{M}(ω)| is maximum amplitude of voltage at each frequency as delivered by a chosen waveform to the fibrous capsule formation and capsular contracture target pathway structure.

An embodiment according to the present invention comprises a pulse burst envelope having a high spectral density, so that the effect of therapy upon the relevant dielectric pathways, such as, cellular membrane receptors, ion binding to cellular enzymes and general transmembrane potential changes, is enhanced. Accordingly by increasing a number of frequency components transmitted to relevant cellular pathways, a large range of biophysical phenomena, such as modulating growth factor and cytokine release and ion binding at regulatory molecules, applicable to known tissue growth mechanisms is accessible. According to an embodiment of the present invention applying a random, or other high spectral density envelope, to a pulse burst envelope of mono-polar or bi-polar rectangular or sinusoidal pulses inducing peak electric fields between about 10⁻⁸ and about 100 mV/cm, produces a greater effect on biological healing processes applicable to both soft and hard tissues.

An embodiment according to the present invention comprises an electromagnetic signal having a pulse burst envelope of spectral density to efficiently couple to physiologically relevant dielectric pathways, such as cellular membrane receptors, ion binding to cellular enzymes, and general transmembrane potential changes. The use of a burst duration which is generally below 100 microseconds for each PRF burst, limits the frequency components that could couple to the relevant dielectric pathways in cells and tissue. An embodiment according to the present invention increases the number of frequency components transmitted to relevant cellular pathways whereby access to a larger range of biophysical phenomena applicable to known healing mechanisms, including enhanced second messenger release, enzyme activity and growth factor and cytokine release can be achieved. By increasing burst duration and applying a random, or other envelope, to the pulse burst envelope of mono-polar or bi-polar rectangular or sinusoidal pulses which induce peak electric fields between 10⁻⁸ and 100 mV/cm, a more efficient and greater effect can be achieved on biological healing processes applicable to both soft and hard tissues in humans, animals and plants.

Another embodiment according to the present invention comprises known cellular responses to weak external stimuli such as heat, light, sound, ultrasound and electromagnetic fields. Cellular responses to such stimuli result in the production of protective proteins, for example, heat shock proteins, which enhance the ability of the cell, tissue, organ to withstand and respond to such external stimuli. Electromagnetic fields configured according to an embodiment of the present invention enhance the release of such compounds thus advantageously providing an improved means to enhance prophylactic protection and wellness of living organisms. After implant surgery there can be physiological deficiencies such as capsular contraction and excessive fibrous capsule formation states that can have a lasting and deleterious effect on an individual's well being and on the proper functioning of an implanted device. Those physiological deficiencies and states can be positively affected on a non-invasive basis by the therapeutic application of waveforms configured according to an embodiment of the present invention. In addition, electromagnetic waveforms configured according to an embodiment of the present invention can have a prophylactic effect on an implant area whereby formation of excessive fibrous tissue may be prevented.

The present invention relates to a therapeutically beneficial method of and apparatus for non-invasive pulsed electromagnetic treatment for enhanced condition, repair and growth of living tissue in animals, humans and plants. This beneficial method operates to selectively change the bioelectromagnetic environment associated with the cellular and tissue environment through the use of electromagnetic means such as PRF generators and applicator heads. More particularly use of electromagnetic means includes the provision of a flux path to a selectable body region, of a succession of EMF pulses having a minimum width characteristic of at least 0.01 microseconds in a pulse burst envelope having between 1 and 100,000 pulses per burst, in which a voltage amplitude envelope of said pulse burst is defined by a randomly varying parameter. Further, the repetition rate of such pulse bursts may vary from 0.01 to 10,000 Hz. Additionally a mathematically-definable parameter can be employed in lieu of said random amplitude envelope of the pulse bursts.

According to an embodiment of the present invention, by applying a random, or other high spectral density envelope, to a pulse burst envelope of mono-polar or bi-polar rectangular or sinusoidal pulses which induce peak electric fields between 10⁻⁸ and 100 millivolts per centimeter (mV/cm), a more efficient and greater effect can be achieved on biological healing processes applicable to both soft and hard tissues in humans, animals and plants. A pulse burst envelope of higher spectral density can advantageously and efficiently couple to physiologically relevant dielectric pathways, such as, cellular membrane receptors, ion binding to cellular enzymes, and general transmembrane potential changes thereby modulating angiogenesis and neovascularization.

An embodiment according to the present invention utilizes a Power Signal to Noise Ratio (“Power SNR”) approach to configure bioeffective waveforms and incorporates miniaturized circuitry and lightweight flexible coils. This advantageously allows a device that utilizes a Power SNR approach, miniaturized circuitry, and lightweight flexible coils, to be completely portable and if desired to be constructed as disposable and if further desired to be constructed as implantable. The lightweight flexible coils can be an integral portion of a positioning device such as surgical dressings, wound dressings, pads, seat cushions, mattress pads, wheelchairs, chairs, and any other garment and structure juxtaposed to living tissue and cells. By advantageously integrating a coil into a positioning device therapeutic treatment can be provided to living tissue and cells in an inconspicuous and convenient manner.

Specifically, broad spectral density bursts of electromagnetic waveforms, configured to achieve maximum signal power within a bandpass of a biological target, are selectively applied to fibrous capsule formation and capsular contracture target pathway structures such as living organs, tissues, cells and molecules that are associated with excessive fibrous capsule formation and capsular contracture. Waveforms are selected using a novel amplitude/power comparison with that of thermal noise in a fibrous capsule formation and capsular contracture target pathway structure. Signals comprise bursts of at least one of sinusoidal, rectangular, chaotic and random wave shapes have frequency content in a range of 0.01 Hz to 100 MHz at 1 to 100,000 bursts per second, with a burst duration from 0.01 to 100 milliseconds, and a burst repetition rate from 0.01 to 1000 bursts/second. Peak signal amplitude at a fibrous capsule formation and capsular contracture target pathway structure such as tissue, lies in a range of 1 μV/cm to 100 mV/cm. Each signal burst envelope may be a random function providing a means to accommodate different electromagnetic characteristics of healing tissue. Preferably the present invention comprises a 20 millisecond pulse burst, repeating at 1 to 10 burst/second and comprising 0.5 to 200 microsecond symmetrical or asymmetrical pulses repeating at 10⁻⁵ to 100 kilohertz within the burst. The burst envelope can be modified 1/f function or any arbitrary function and can be applied at random repetition rates. Fixed repetition rates can also be used between about 0.1 Hz and about 1000 Hz. An induced electric field from about 10⁻⁸ mV/cm to about 100 mV/cm is generated. Another embodiment according to the present invention comprises a 4 millisecond of high frequency sinusoidal waves, such as 27.12 MHz, repeating at 1 to 100 bursts per second. An induced electric field from about 10⁻⁸ mV/cm to about 100 mV/cm is generated. Resulting waveforms can be delivered via inductive or capacitive coupling for 1 to 30 minute treatment sessions delivered according to predefined regimes by which PEMF treatment may be applied for 1 to 12 daily sessions, repeated daily. The treatment regimens for any waveform configured according to the instant invention may be fully automated. The number of daily treatments may be programmed to vary on a daily basis according to any predefined protocol.

According to yet another embodiment of the present invention by applying a high spectral density voltage envelope as a modulating or pulse-burst defining parameter, power requirements for such amplitude modulated pulse bursts can be significantly lower than that of an unmodulated or continuous pulse burst containing pulses within a similar carrier frequency range. This is due to a substantial reduction in duty cycle within repetitive burst trains brought about by imposition of an irregular amplitude and preferably a random amplitude onto what would otherwise be a substantially uniform pulse burst envelope. Accordingly, the dual advantages, of enhanced transmitted dosimetry to the relevant dielectric pathways and of decreased power requirement are achieved.

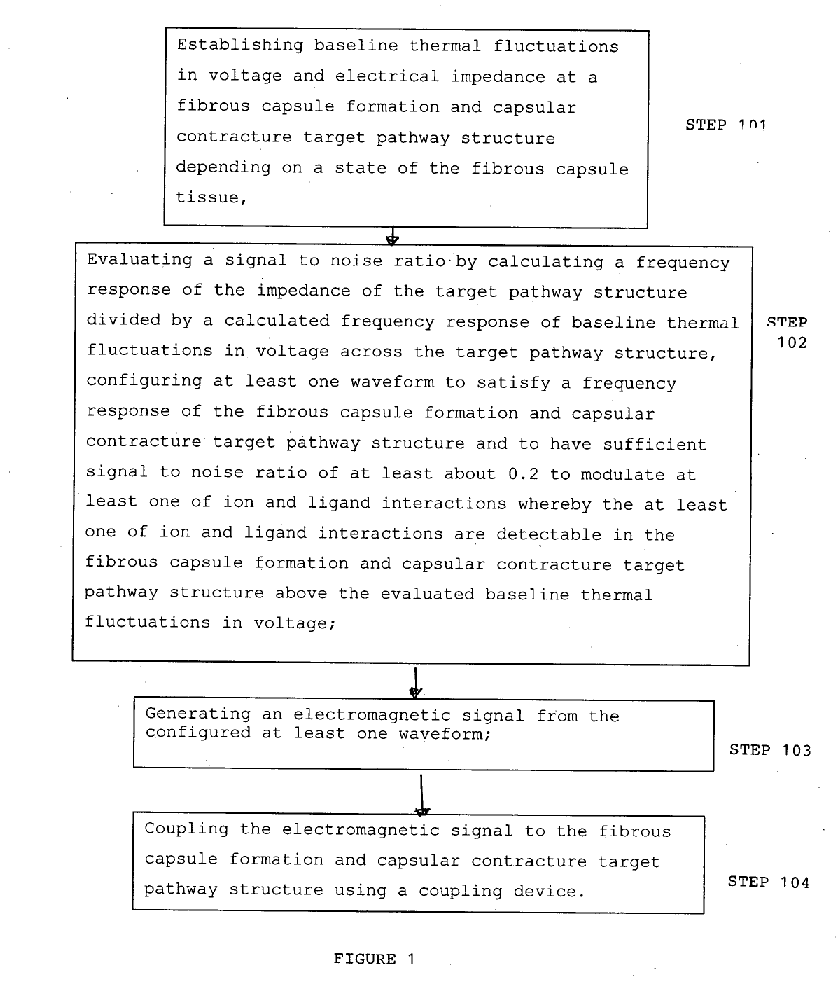

Referring to FIG. 1 wherein FIG. 1 is a flow diagram of a method for generating electromagnetic signals to be coupled to a fibrous capsule formation and capsular contracture target pathway structure according to an embodiment of the present invention, a fibrous capsule formation and capsular contracture target pathway structure such as ions and ligands, is identified. Establishing a baseline background activity such as baseline thermal fluctuations in voltage and electrical impedance, at the fibrous capsule formation and capsular contracture target pathway structure by determining a state of at least one of a cell and a tissue at the fibrous capsule formation and capsular contracture target pathway structure, wherein the state is at least one of resting, growing, replacing, and responding to injury. (STEP 101) The state of the at least one of a cell and a tissue is determined by its response to injury or insult. Configuring at least one waveform to have sufficient signal to noise ratio to modulate at least one of ion and ligand interactions whereby the at least one of ion and ligand interactions are detectable in the fibrous capsule formation and capsular contracture target pathway structure above the established baseline thermal fluctuations in voltage and electrical impedance. The EMF signal can be generated by using at least one waveform configured by applying a mathematical model such as an equation, formula, or function having at least one waveform parameter that satisfies an SNR or Power SNR mathematical model of at least about 0.2, to modulate at least one of ion and ligand interactions whereby the at least one of ion and ligand interactions are detectable in a fibrous capsule formation and capsular contracture target pathway structure above baseline thermal fluctuations in voltage and electrical impedance at the fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure (STEP 102). Repetitively generating an electromagnetic signal from the configured at least one waveform (STEP 103). Coupling the electromagnetic signal to the fibrous capsule formation and capsular contracture target pathway structure using a coupling device (STEP 104). The generated electromagnetic signals can be coupled for therapeutic and prophylactic purposes. The coupling enhances a stimulus that cells and tissues react to in a physiological meaningful manner for example, an increase in angiogenesis, neovascularization and vascularogenesis or other physiological effects related to the improvement of excessive fibrous tissue or capsular contracture. Application of electromagnetic signals using an embodiment according to the present invention is extremely safe and efficient since the application of electromagnetic signals configured according to the present invention is non-invasive and athermal.

In the present invention, a generated electromagnetic signal is comprised of a burst of arbitrary waveforms having at least one waveform parameter that includes a plurality of frequency components ranging from about 0.01 Hz to about 100 MHz wherein the plurality of frequency components satisfies a Power SNR model. A repetitive electromagnetic signal can be generated for example inductively or capacitively, from the configured at least one waveform. The electromagnetic signal is coupled to a fibrous capsule formation and capsular contracture target pathway structure such as ions and ligands by output of a coupling device such as an electrode or an inductor, placed in close proximity to the fibrous capsule formation and capsular contracture target pathway structure using a positioning device. The coupling enhances modulation of binding of ions and ligands to regulatory molecules, tissues, cells, and organs. According to an embodiment of the present invention EMF signals configured using SNR analysis to match the bandpass of a second messenger whereby the EMF signals can act as a first messenger to modulate biochemical cascades such as production of cytokines, Nitric Oxide, Nitric Oxide Synthase and growth factors that are related to tissue growth and repair. A detectable E field amplitude is produced within a frequency response of Ca²⁺ binding.

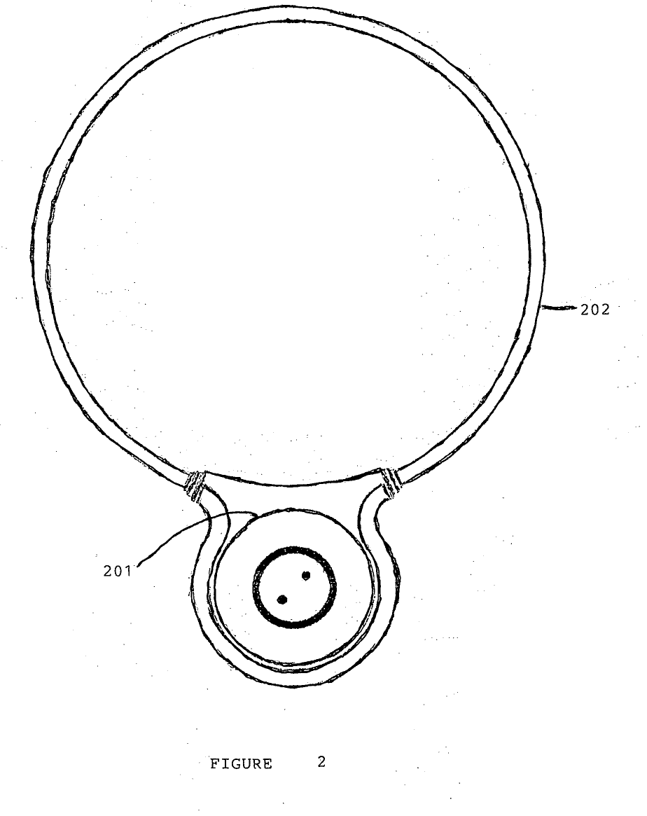

FIG. 2 illustrates an embodiment of an apparatus according to the present invention. The apparatus is constructed to be self-contained, lightweight, and portable. A miniature control circuit 201 is connected to a generating device such as an electrical coil 202. The miniature control circuit 201 is constructed in a manner that applies a mathematical model that is used to configure waveforms. The configured waveforms have to satisfy a Power SNR model so that for a given and known fibrous capsule formation and capsular contracture target pathway structure, it is possible to choose waveform parameters that satisfy a frequency response of the fibrous capsule formation and capsular contracture target pathway structure and Power SNR of at least about 0.2 to modulate at least one of ion and ligand interactions whereby the at least one of ion and ligand interactions are detectable in a fibrous capsule formation and capsular contracture target pathway structure above baseline thermal fluctuations in voltage and electrical impedance at the fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure. An embodiment according to the present invention applies a mathematical model to induce a time-varying magnetic field and a time-varying electric field in a fibrous capsule formation and capsular contracture target pathway structure such as ions and ligands, comprising about 0.001 to about 100 msec bursts of about 1 to about 100 microsecond rectangular pulses, having a burst duration of about 0.01 to 100,000 microseconds and repeating at about 0.1 to about 100 pulses per second. Peak amplitude of the induced electric field is between about 1 uV/cm and about 100 mV/cm, that can be constant or varied according to a mathematical function, for example a modified 1/f function where f=frequency. A waveform configured using an embodiment according to the present invention may be applied to a fibrous capsule formation and capsular contracture target pathway structure such as ions and ligands, preferably for a total exposure time of under 1 minute to 240 minutes daily. However other exposure times can be used. Waveforms configured by the miniature control circuit 201 are directed to a generating device 202 such as electrical coils. Preferably, the generating device 202 is a conformable coil for example pliable, comprising one or more turns of electrically conducting wire in a generally circular or oval shape however other shapes can be used. The generating device 202 delivers a pulsing magnetic field configured according to a mathematical model that can be used to provide treatment to a fibrous capsule formation and capsular contracture target pathway structure such as mammary tissue. The miniature control circuit applies a pulsing magnetic field for a prescribed time and can automatically repeat applying the pulsing magnetic field for as many applications as are needed in a given time period, for example 12 times a day. The miniature control circuit can be configured to be programmable applying pulsing magnetic fields for any time repetition sequence. An embodiment according to the present invention can be positioned to treat fibrous capsule tissue by being incorporated with a positioning device such as a bandage, a vest, a brassiere, or an anatomical support thereby making the unit self-contained. Coupling a pulsing magnetic field to a fibrous capsule formation and capsular contracture target pathway structure such as ions and ligands, therapeutically and prophylactically reduces inflammation thereby reducing pain and promotes healing in treatment areas. When electrical coils are used as the generating device 202, the electrical coils can be powered with a time varying magnetic field that induces a time varying electric field in a fibrous capsule formation and capsular contracture target pathway structure according to Faraday's law. An electromagnetic signal generated by the generating device 202 can also be applied using electrochemical coupling, wherein electrodes are in direct contact with skin or another outer electrically conductive boundary of a fibrous capsule formation and capsular contracture target pathway structure. Yet in another embodiment according to the present invention, the electromagnetic signal generated by the generating device 202 can also be applied using electrostatic coupling wherein an air gap exists between a generating device 202 such as an electrode and a fibrous capsule formation and capsular contracture target pathway structure such as ions and ligands. An advantage of the present invention is that its ultra lightweight coils and miniaturized circuitry allow for use with common physical therapy treatment modalities, and at any location for which tissue growth, pain relief, and tissue and organ healing is desired. An advantageous result of application of the present invention is that tissue growth, repair, and maintenance can be accomplished and enhanced anywhere and at anytime. Yet another advantageous result of application of the present invention is that growth, repair, and maintenance of molecules, cells, tissues, and organs can be accomplished and enhanced anywhere and at anytime. Another embodiment according to the present invention delivers PEMF for application to capsular contracture and excessive fibrous capsule tissue that resulted from implant surgery such as breast augmentation.

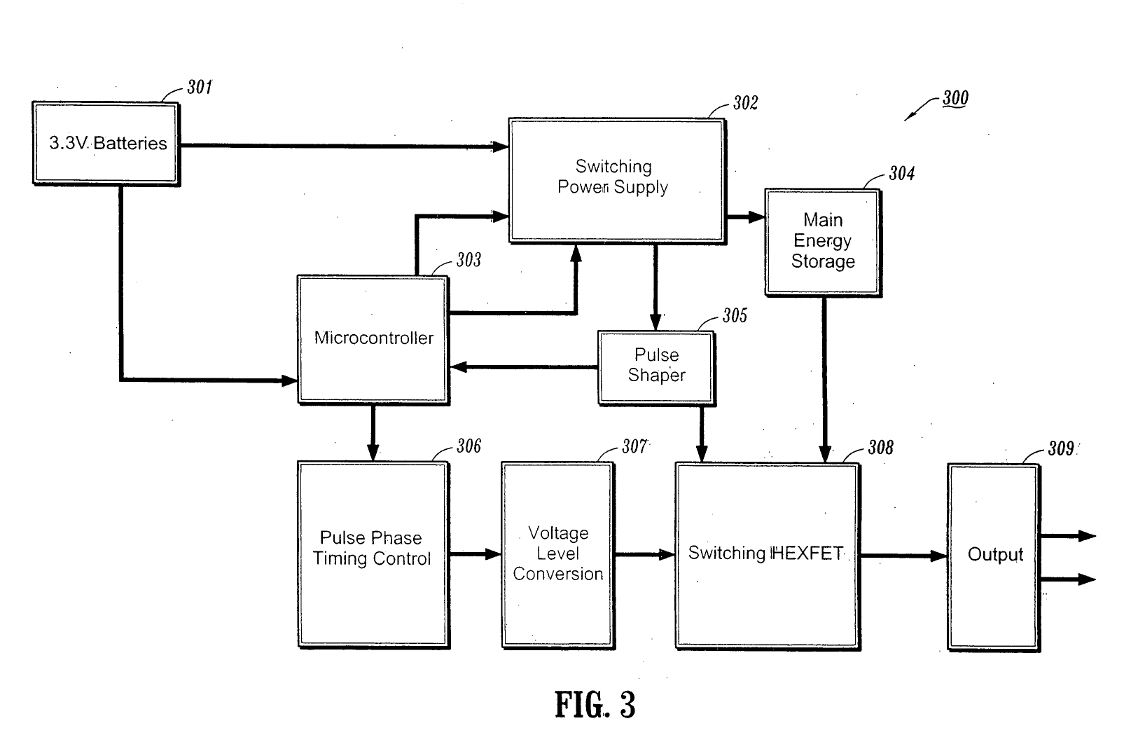

FIG. 3 depicts a block diagram of an embodiment according to the present invention of a miniature control circuit 300. The miniature control circuit 300 produces waveforms that drive a generating device such as wire coils described above in FIG. 2. The miniature control circuit can be activated by any activation means such as an on/off switch. The miniature control circuit 300 has a power source such as a lithium battery 301. Preferably the power source has an output voltage of 3.3 V but other voltages can be used. In another embodiment according to the present invention the power source can be an external power source such as an electric current outlet such as an AC/DC outlet, coupled to the present invention for example by a plug and wire. A switching power supply 302 controls voltage to a micro-controller 303. Preferably the micro-controller 303 uses an 8 bit 4 MHz micro-controller 303 but other bit MHz combination micro-controllers may be used. The switching power supply 302 also delivers current to storage capacitors 304. Preferably the storage capacitors 304 having a 220 uF output but other outputs can be used. The storage capacitors 304 allow high frequency pulses to be delivered to a coupling device such as inductors (Not Shown). The micro-controller 303 also controls a pulse shaper 305 and a pulse phase timing control 306. The pulse shaper 305 and pulse phase timing control 306 determine pulse shape, burst width, burst envelope shape, and burst repetition rate. In an embodiment according to the present invention the pulse shaper 305 and phase timing control 306 are configured such that the waveforms configured are detectable above background activity at a fibrous capsule formation and capsular contracture target pathway structure by satisfying at least one of a SNR and Power SNR mathematical model. An integral waveform generator, such as a sine wave or arbitrary number generator can also be incorporated to provide specific waveforms. A voltage level conversion sub-circuit 307 controls an induced field delivered to a fibrous capsule formation and capsular contracture target pathway structure. A switching Hexfet 308 allows pulses of randomized amplitude to be delivered to output 309 that routes a waveform to at least one coupling device such as an inductor. The micro-controller 303 can also control total exposure time of a single treatment of a fibrous capsule formation and capsular contracture target pathway structure such as a molecule, cell, tissue, and organ. The miniature control circuit 300 can be constructed to be programmable and apply a pulsing magnetic field for a prescribed time and to automatically repeat applying the pulsing magnetic field for as many applications as are needed in a given time period, for example 10 times a day. Preferably treatments times of about 1 minutes to about 30 minutes are used.

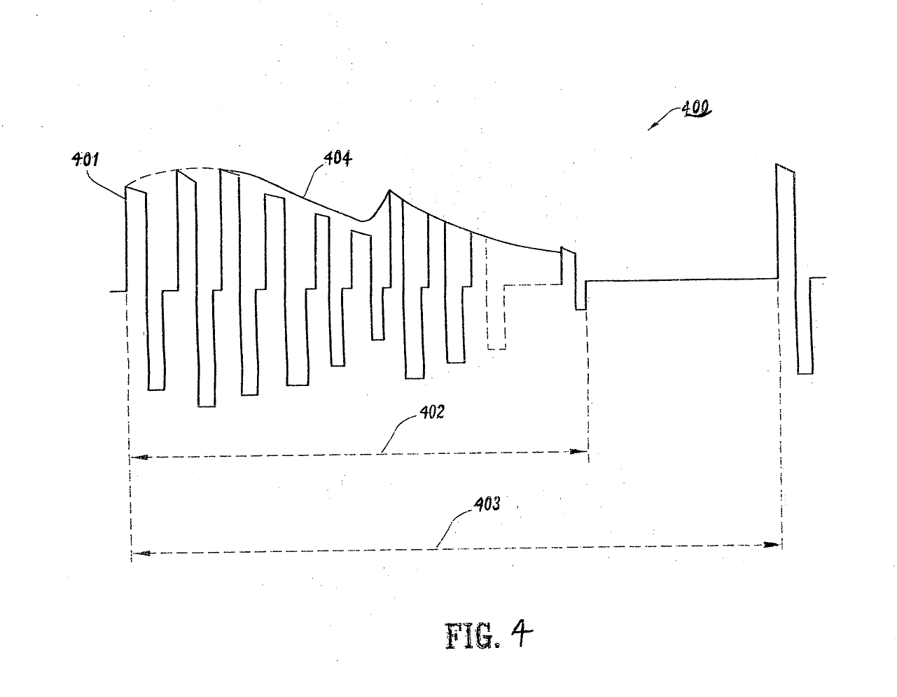

Referring to FIG. 4 an embodiment according to the present invention of a waveform 400 is illustrated. A pulse 401 is repeated within a burst 402 that has a finite duration 403 alternatively referred to as width 403. The duration 403 is such that a duty cycle which can be defined as a ratio of burst duration to signal period is between about 1 to about 10⁻⁵. Preferably pseudo rectangular 10 microsecond pulses for pulse 401 applied in a burst 402 for about 10 to about 50 msec having a modified 1/f amplitude envelope 404 and with a finite duration 403 corresponding to a burst period of between about 0.1 and about 10 seconds are utilized.

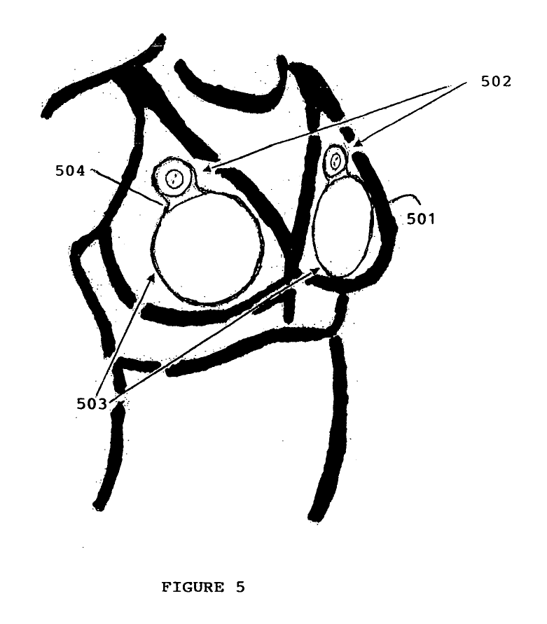

FIG. 5 illustrates an embodiment of an apparatus according to the present invention. A garment 501 such as a brassiere is constructed out of materials that are lightweight and portable such as nylon but other materials can be used. A miniature control circuit 502 is coupled to a generating device such as an electrical coil 503. Preferably the miniature control circuit 502 and the electrical coil 503 are constructed in a manner as described above in reference to FIG. 2. The miniature control circuit and the electrical coil can be connected with a connecting means such as a wire 504. The connection can also be direct or wireless. The electrical coil 503 is integrated into the garment 501 such that when a user wears the garment 501, the electrical coil is positioned near an excessive fibrous capsule formation location or capsular contracture location of the user. An advantage of the present invention is that its ultra lightweight coils and miniaturized circuitry allow for the garment 501 to be completely self-contained, portable, and lightweight. An additionally advantageous result of the present invention is that the garment 501 can be constructed to be inconspicuous when worn and can be worn as an outer garment such as a shirt or under other garments, so that only the user will know that the garment 501 is being worn and treatment is being applied. Use with common physical therapy treatment modalities, and at any excessive fibrous capsule location or capsular contracture location for which pain relief, and tissue and organ healing is easily obtained. An advantageous result of application of the present invention is that tissue growth, repair, and maintenance can be accomplished and enhanced anywhere and at anytime. Yet another advantageous result of application of the present invention is that growth, repair, and maintenance of molecules, cells, tissues, and organs can be accomplished and enhanced anywhere and at anytime. Another embodiment according to the present invention delivers PEMF for application to fibrous capsules.

It is further intended that any other embodiments of the present invention that result from any changes in application or method of use or operation, method of manufacture, shape, size or material which are not specified within the detailed written description or illustrations and drawings contained herein, yet are considered apparent or obvious to one skilled in the art, are within the scope of the present invention.

The process of the invention will now be described with reference to the following illustrative examples.

### EXAMPLE 1

The Power SNR approach for PMF signal configuration has been tested experimentally on calcium dependent myosin phosphorylation in a standard enzyme assay. The cell-free reaction mixture was chosen for phosphorylation rate to be linear in time for several minutes, and for sub-saturation Ca²⁺ concentration. This opens the biological window for Ca²⁺/CaM to be EMF-sensitive. This system is not responsive to PMF at levels utilized in this study if Ca is at saturation levels with respect to CaM, and reaction is not slowed to a minute time range. Experiments were performed using myosin light chain (“MLC”) and myosin light chain kinase (“MLCK”) isolated from turkey gizzard. A reaction mixture consisted of a basic solution containing 40 mM Hepes buffer, pH 7.0; 0.5 mM magnesium acetate; 1 mg/ml bovine serum albumin, 0.1% (w/v) Tween80; and 1 mM EGTA12. Free Ca²⁺ was varied in the 1-7 μM range. Once Ca²⁺ buffering was established, freshly prepared 70 nM CaM, 160 nM MLC and 2 nM MLCK were added to the basic solution to form a final reaction mixture. The low MLC/MLCK ratio allowed linear time behavior in the minute time range. This provided reproducible enzyme activities and minimized pipetting time errors.

The reaction mixture was freshly prepared daily for each series of experiments and was aliquoted in 100 μL portions into 1.5 ml Eppendorf tubes. All Eppendorf tubes containing reaction mixture were kept at 0° C. then transferred to a specially designed water bath maintained at 37±0.1° C. by constant perfusion of water prewarmed by passage through a Fisher Scientific model 900 heat exchanger. Temperature was monitored with a thermistor probe such as a Cole-Parmer model 8110-20, immersed in one Eppendorf tube during all experiments. Reaction was initiated with 2.5 μM 32P ATP, and was stopped with Laemmli Sample Buffer solution containing 30 μM EDTA. A minimum of five blank samples were counted in each experiment. Blanks comprised a total assay mixture minus one of the active components Ca²⁺, CaM, MLC or MLCK. Experiments for which blank counts were higher than 300 cpm were rejected. Phosphorylation was allowed to proceed for 5 min and was evaluated by counting 32P incorporated in MLC using a TM Analytic model 5303 Mark V liquid scintillation counter.

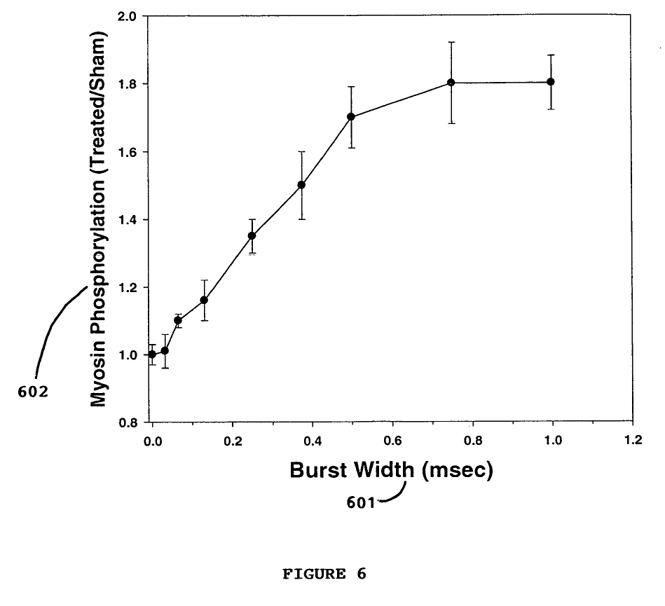

The signal comprised repetitive bursts of a high frequency waveform. Amplitude was maintained constant at 0.2 G and repetition rate was 1 burst/sec for all exposures. Burst duration varied from 65 μsec to 1000 μsec based upon projections of Power SNR analysis which showed that optimal Power SNR would be achieved as burst duration approached 500 μsec. The results are shown in FIG. 6 wherein burst width 601 in msec is plotted on the x-axis and Myosin Phosphorylation 602 as treated/sham is plotted on the y-axis. It can be seen that the PMF effect on Ca²⁺ binding to CaM approaches its maximum at approximately 500 μsec, just as illustrated by the Power SNR model.

These results confirm that a PMF signal, configured according to an embodiment of the present invention, would maximally increase myosin phosphorylation for burst durations sufficient to achieve optimal Power SNR for a given magnetic field amplitude.

### EXAMPLE 2

According to an embodiment of the present invention use of a Power SNR model was further verified in an in vivo wound repair model. A rat wound model has been well characterized both biomechanically and biochemically, and was used in this study. Healthy, young adult male Sprague Dawley rats weighing more than 300 grams were utilized.

The animals were anesthetized with an intraperitoneal dose of Ketamine 75 mg/kg and Medetomidine 0.5 mg/kg. After adequate anesthesia had been achieved, the dorsum was shaved, prepped with a dilute betadine/alcohol solution, and draped using sterile technique. Using a #10 scalpel, an 8-cm linear incision was performed through the skin down to the fascia on the dorsum of each rat. The wound edges were bluntly dissected to break any remaining dermal fibers, leaving an open wound approximately 4 cm in diameter. Hemostasis was obtained with applied pressure to avoid any damage to the skin edges. The skin edges were then closed with a 4-0 Ethilon running suture. Post-operatively, the animals received Buprenorphine 0.1-0.5 mg/kg, intraperitoneal. They were placed in individual cages and received food and water ad libitum.

PMF exposure comprised two pulsed radio frequency waveforms. The first was a standard clinical PRF signal comprising a 65 μsec burst of 27.12 MHz sinusoidal waves at 1 Gauss amplitude and repeating at 600 bursts/sec. The second was a PRF signal reconfigured according to an embodiment of the present invention. For this signal burst duration was increased to 2000 μsec and the amplitude and repetition rate were reduced to 0.2 G and 5 bursts/sec respectively. PRF was applied for 30 minutes twice daily.

Tensile strength was performed immediately after wound excision. Two 1 cm width strips of skin were transected perpendicular to the scar from each sample and used to measure the tensile strength in kg/mm2. The strips were excised from the same area in each rat to assure consistency of measurement. The strips were then mounted on a tensiometer. The strips were loaded at 10 mm/min and the maximum force generated before the wound pulled apart was recorded. The final tensile strength for comparison was determined by taking the average of the maximum load in kilograms per mm2 of the two strips from the same wound.

The results showed average tensile strength for the 65 μsec 1 Gauss PRF signal was 19.3±4.3 kg/mm2 for the exposed group versus 13.0±3.5 kg/mm2 for the control group (p<0.01), which is a 48% increase. In contrast, the average tensile strength for the 2000 μsec 0.2 Gauss PRF signal, configured according to an embodiment of the present invention using a Power SNR model was 21.2±5.6 kg/mm2 for the treated group versus 13.7±4.1 kg/mm2 (p<0.01) for the control group, which is a 54% increase. The results for the two signals were not significantly different from each other.

These results demonstrate that an embodiment of the present invention allowed a new PRF signal to be configured that could be produced with significantly lower power. The PRF signal configured according to an embodiment of the present invention, accelerated wound repair in the rat model in a low power manner versus that for a clinical PRF signal which accelerated wound repair but required more than two orders of magnitude more power to produce.

### EXAMPLE 3

This example illustrates the effects of PMF stimulation of a T-cell receptor with cell arrest and thus behave as normal T-lymphocytes stimulated by antigens at the T-cell receptor such as anti-CD3.

In bone healing, results have shown that both 60 Hz and PEMF fields decrease DNA synthesis of Jurkat cells, as is expected since PMF interacts with the T-cell receptor in the absence of a costimulatory signal. This result is consistent with an anti-inflammatory response, as has been observed in clinical applications of PMF stimuli. The PEMF signal is more effective. A dosimetry analysis performed according to an embodiment of the present invention demonstrates why both signals are effective and why PEMF signals have a greater effect than 60 Hz signals on Jurkat cells in the most EMF-sensitive growth stage.

Comparison of dosimetry from the two signals employed involves evaluation of the ratio of the Power spectrum of the thermal noise voltage that is Power SNR, to that of the induced voltage at the EMF-sensitive target pathway structure. The target pathway structure used is ion binding at receptor sites on Jurkat cells suspended in 2 mm of culture medium. The average peak electric field at the binding site from a PEMF signal comprising 5 msec burst of 200 μsec pulses repeating at 15/sec was 1 mV/cm, while for a 60 Hz signal the average peak electric field was 100 μV/cm.

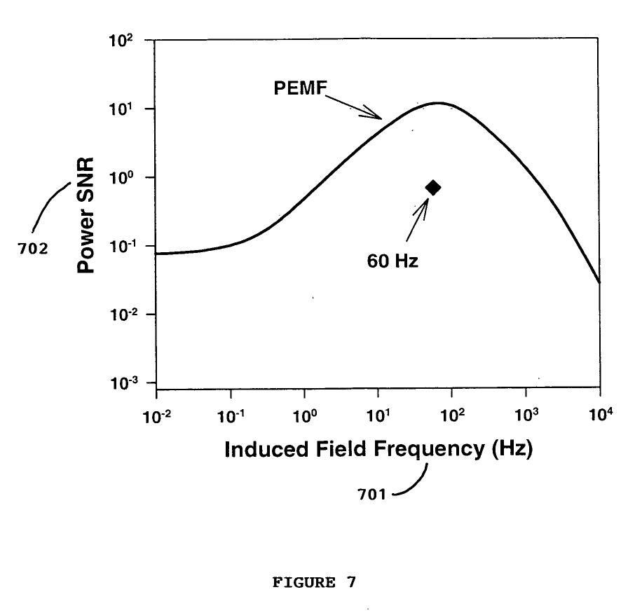

FIG. 7, is a graph of results wherein Induced Field Frequency 701 in Hz is shown on the x-axis and Power SNR 702 is shown on the y-axis. FIG. 7 illustrates that both signals have sufficient Power spectrum that is Power SNR ≧1, to be detected within a frequency range of binding kinetics. However, maximum Power SNR for the PEMF signal is significantly higher than that of the 60 Hz signal. This is due to a PEMF signal having many frequency components falling within a bandpass of the target pathway structure. The single frequency component of a 60 Hz signal lies at the mid-point of the bandpass of a target pathway structure. The Power SNR calculation that was used in this example is dependent upon τᵢₒₙ which is obtained from the rate constant for ion binding. Had this calculation been performed a priori it would have concluded that both signals satisfied basic detectability requirements and could modulate an EMF-sensitive ion binding pathway at the start of a regulatory cascade for DNA synthesis in these cells. The previous examples illustrate that utilizing the rate constant for Ca/CaM binding could lead to successful projections for bioeffective EMF signals in a variety of systems.

### EXAMPLE 4

In this example six patients who had developed capsular contracture after receiving bilateral breast implants were treated with a special support brassiere having embedded coils located in each cup and a generator for each coil located in a special pocket in the strap above each cup as described in FIG. 5 above. PEMF signals generated by the apparatus configured according to an embodiment of the present invention comprised a repetitive burst of radio frequency sinusoidal waves configured according to an embodiment of the present invention. The PEMF signal induced a peak electric field in a range of 1 to 10 mV/cm. All patients were provided a regimen that comprised six thirty minute sessions for days 1 to 3 post implant, four sessions for days 4 to 6 post implant, and two sessions for all subsequent days. Clinical evaluation demonstrated that by day 7 the fibrous capsule was significantly softer and patients reported significantly less pain and discomfort than prior to the treatment. Clinical evaluations at one and three months post PEMF treatment revealed significant resolution of the fibrous capsule and its corresponding symptoms.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

What is claimed is:

1) An apparatus comprising:
an electromagnetic signal generating means for emitting signals comprising bursts of at least one of sinusoidal, rectangular, chaotic, and random waveforms, having a frequency content in a range of about 0.01 Hz to about 100 MHz at about 1 to about 100,000 waveforms per second, having a burst duration from about 1 usec to about 100 msec, and having a burst repetition rate from about 0.01 to about 1000 bursts/second, wherein the waveforms are adapted to a frequency response of a fibrous capsule formation and capsular contracture target pathway structure and to have sufficient signal to noise ratio of at least about 0.2 in respect of a given fibrous capsule formation and capsular contracture target pathway structure to modulate at least one of ion and ligand interactions in that fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure,
an electromagnetic signal coupling means wherein the coupling means comprises at least one of an inductive coupling means and a capacitive coupling means, connected to the electromagnetic signal generating means for delivering the electromagnetic signal to the fibrous capsule formation and capsular contracture target pathway structure, and
a garment wherein the electromagnetic signal generating means and electromagnetic signal coupling means are incorporated into the garment.

2) The apparatus of claim 1, wherein the signals comprise about 0.001 to about 100 msec bursts repeating at about 0.1 to about 100 pulses per second of about 1 to about 100 microsecond rectangular pulses.

3) The apparatus of claim 1, configured for providing an emitted signal having an peak signal amplitude at a fibrous capsule formation and capsular contracture target pathway structure in a range of about 1 μV/cm to about 100 mV/cm.

4) The apparatus of claim 1, wherein each signal burst envelope is a random function for providing a means to accommodate different electromagnetic characteristics of healing tissue.

5) The apparatus of claim 1, wherein the apparatus is configured for emitting a 20 millisecond pulse burst comprising about 0.1 microsecond to about 20 microsecond at least one of symmetrical and asymmetrical pulses repeating at about 1 to about 100 KHz within the burst.

6) The apparatus of claim 1, wherein the apparatus is configured for emitting an about 1 millisecond to an about 5 millisecond burst of 27.12 MHz sinusoidal waves repeating at about 1 to about 100 bursts/sec.

7) The apparatus of claim 1, wherein the garment includes at least one of a brassiere, a surgical dressing, an anatomical support.

8) An apparatus comprising:
A waveform configuration means for configuring at least one waveform to a frequency response of a fibrous capsule formation and capsular contracture target pathway structure and to have sufficient signal to noise ratio or power signal to noise ratio of at least about 0.2, to modulate at least one of ion and ligand interactions whereby the at least one of ion and ligand interactions are detectable in a fibrous capsule formation and capsular contracture target pathway structure above baseline thermal fluctuations in voltage and electrical impedance at the fibrous capsule formation and capsular contracture target pathway structure, wherein the signal to noise ratio is evaluated by calculating a frequency response of the impedance of the target path structure divided by a calculated frequency response of baseline thermal fluctuations in voltage across the target path structure;
A coupling device connected to the waveform configuration means by at least one connecting means for generating an electromagnetic signal from the configured at least one waveform and for coupling the electromagnetic signal to the fibrous capsule formation and capsular contracture target pathway structure whereby the at least one of ion and ligand interactions are modulated; and
A garment incorporating the waveform configuration means, the at least one connecting means, and the coupling device.

9) The apparatus of claim 8, wherein the at least one of ion and ligand interactions includes at least one of calcium ion binding and binding of calcium ions to calmodulin.

10) The apparatus of claim 8, wherein the configuration means includes a configuration means for configuring at least one waveform having at least one of a frequency component parameter that configures said at least one waveform to be between about 0.01 Hz and about 100 MHz, a burst amplitude envelope parameter that follows an arbitrary amplitude function, a burst amplitude envelope parameter that follows a defined amplitude function, a burst width parameter that varies at each repetition according to an arbitrary width function, a burst width parameter that varies at each repetition according to a defined width function, a peak induced electric field parameter varying between about 1 μV/cm and about 100 mV/cm in said fibrous capsule formation and capsular contracture target pathway structure, and a peak induced magnetic field parameter varying between about 1 μT and about 0.1 T in said fibrous capsule formation and capsular contracture target pathway structure.

11) The apparatus of claim 10, wherein said defined amplitude function includes at least one of a 1/frequency function, a logarithmic function, a chaotic function and an exponential function.

12) The apparatus of claim 8, wherein said coupling device includes at least one of an inductive generating coupling device, a capacitive generating coupling device, an inductor, and an electrode.

13) The apparatus of claim 8, wherein at least one of said waveform configuration means, connecting means, and coupling device is at least one of portable, disposable, implantable, and wireless.

14) The apparatus of claim 8, wherein the garment includes at least one of a brassiere, a surgical dressing, an anatomical support.

15) A method comprising:
Establishing baseline thermal fluctuations in voltage and electrical impedance at a fibrous capsule formation and capsular contracture target pathway structure depending on a state of the fibrous capsule tissue,
Evaluating a signal to noise ratio by calculating a frequency response of the impedance of the target pathway structure divided by a calculated frequency response of baseline thermal fluctuations in voltage across the target pathway structure,
Configuring at least one waveform to a frequency response of the fibrous capsule formation and capsular contracture target pathway structure and to have sufficient signal to noise ratio of at least about 0.2 to modulate at least one of ion and ligand interactions whereby the at least one of ion and ligand interactions are detectable in the fibrous capsule formation and capsular contracture target pathway structure above the evaluated baseline thermal fluctuations in voltage;
Generating an electromagnetic signal from the configured at least one waveform; and
Coupling the electromagnetic signal to the fibrous capsule formation and capsular contracture target pathway structure using a coupling device.

16) The method of claim 15, wherein the step of configuring at least one waveform to have sufficient signal to noise ratio of at least about 0.2 to modulate at least one of ion and ligand interactions includes configuring at least one waveform to have sufficient signal to noise ratio to modulate calcium ion binding.

17) The method of claim 15, wherein the step of configuring at least one waveform to have sufficient signal to noise ratio of at least about 0.2 to modulate at least one of ion and ligand interactions includes configuring at least one waveform to have sufficient signal to noise ratio to modulate binding of calcium ions to calmodulin.

18) The method of claim 15, wherein the step of configuring at least one waveform to have sufficient signal to noise ratio of at least about 0.2 to modulate at least one of ion and ligand interactions includes configuring at least one waveform to have sufficient signal to noise ratio to match a bandpass of a second messenger at a fibrous capsule formation and capsular contracture target pathway structure whereby the second messenger modulates biochemical cascades related to tissue growth and repair.

19) The method of claim 15, wherein the step of establishing baseline thermal fluctuations in voltage and electrical impedance at a fibrous capsule formation and capsular contracture target pathway structure includes establishing baseline thermal fluctuations in voltage and electrical impedance at least one of a fibrous capsule molecule, a fibrous capsule cell, a fibrous capsule tissue, and a fibrous capsule organ.

20) The method of claim 15, wherein the step of coupling the electromagnetic signal to the fibrous capsule formation and capsular contracture target pathway structure using a coupling device includes coupling the electromagnetic signal to the fibrous capsule formation and capsular contracture target pathway structure using at least one of an inductive generating coupling device, a capacitive generating coupling device, an inductor, and an electrode.

21) The method of claim 15, wherein the step of coupling the electromagnetic signal to the fibrous capsule formation and capsular contracture target pathway structure includes coupling the electromagnetic signal to the fibrous capsule formation and capsular contracture target pathway structure to enhance the production of second messengers at the fibrous capsule formation and capsular contracture target pathway structure.

22) The method of claim 21, wherein the step of coupling the electromagnetic signal to the fibrous capsule formation and capsular contracture target pathway structure to enhance the production of second messengers at the fibrous capsule formation and capsular contracture target pathway structure includes coupling the electromagnetic signal to the fibrous capsule formation and capsular contracture target pathway structure to enhance the production of Nitric Oxide at the fibrous capsule formation and capsular contracture target pathway structure.

23) The method of claim 15, wherein the step of coupling the electromagnetic signal to the fibrous capsule formation and capsular contracture target pathway structure includes coupling the electromagnetic signal to the fibrous capsule formation and capsular contracture target pathway structure to enhance the production of at least one of growth factors and cytokines at the fibrous capsule formation and capsular contracture target pathway structure.

24) The method of claim 15, wherein the step of coupling the electromagnetic signal to the fibrous capsule formation and capsular contracture target pathway structure includes coupling the electromagnetic signal to the fibrous capsule formation and capsular contracture target pathway structure to enhance modulation of binding of at least one of ions and ligands to at least one of regulatory molecules, tissues, cells, and organs.

25) The method of claim 15, wherein the step of coupling the electromagnetic signal to the fibrous capsule formation and capsular contracture target pathway structure includes coupling the electromagnetic signal to the fibrous capsule formation and capsular contracture target pathway structure to provide treatment for at least one of excessive fibrous capsule formation and capsular contracture.
